(19) United States
(12) Patent Application Publication
(10) Pub. No.: US 2025/0001379 A1
(43) Pub. Date: Jan. 2, 2025

(54) REACTOR AND METHOD FOR SYNTHESIZING METHANOL

(71) Applicants: CLARIANT INTERNATIONAL LTD., Muttenz (CH); L'AIR LIQUIDE, SOCIÉTÉ ANONYME POUR L'ETUDE ET L'EXPLOITATION DES PROCÉDÉS GEORGES CLAUDE, Paris (FR)

(72) Inventors: Stéphane HAAG, Frankfurt am Main (DE); Nga Thi Quynh DO, Frankfurt am Main (DE); Veronika GRONEMANN, Frankfurt am Main (DE); Tobias OELMANN, Frankfurt am Main (DE); Markus REICHINGER, Bruckmühl (DE); Stephan J. REITMEIER, Bruckmühl (DE); Heiner SCHWARZ, München (DE)

(73) Assignees: CLARIANT INTERNATIONAL LTD., Muttenz (CH); L'AIR LIQUIDE, SOCIÉTÉ ANONYME POUR L'ETUDE ET L'EXPLOITATION DES PROCÉDÉS GEORGES CLAUDE, Paris (FR)

(21) Appl. No.: 18/693,278

(22) PCT Filed: Sep. 20, 2022
(86) PCT No.: PCT/EP2022/076105

§ 371 Date: Mar. 19, 2024

(30) Foreign Application Priority Data
Sep. 23, 2021 (EP) 21198533.8

(51) Int. Cl. B01J 8/04; B01J 8/06; C07C 1/04

(52) U.S. Cl. CPC B01J 8/0453; B01J 8/067; C07C 1/041; B01J 2208/00336; B01J 2208/00628

(57) ABSTRACT

A reactor for the catalytic production of methanol having two or more reaction zones arranged and surrounded by a heat exchange fluid. The reaction zones are preferably reaction tubes, and each reaction zone has at least one cleaning layer and at least one catalyst layer, where the cleaning layer is arranged upstream, and the catalyst layer is arranged downstream. A method for the catalytic production of methanol from synthesis gas using the reactor is also described.

The invention relates to a reactor for the catalytic production of methanol, and a method for the catalytic production of methanol from synthesis gas.

### BACKGROUND OF THE INVENTION

Methods for producing methanol through catalytic conversion of synthesis gas containing hydrogen and carbon oxides have been known to the expert community for a long time. Thus, a method for producing methanol is described in Ullmann's Encyclopedia of Industrial Chemistry, Sixth Edition, 1998 Electronic Release, chapter “Methanol”, subchapter 5.2 “Synthesis”. The known synthesis of methanol from synthesis gas, which usually comprises carbon monoxide, carbon dioxide and hydrogen, can be described by the following equations:

CO+2H₂≈CH₃OH; 1)

CO₂+3H₂≈CH₃OH+H₂O; 2)

CO+H₂O≈H₂+CO₂ 3)

These three reactions are exothermic. In reactions 1 and 2 methanol is formed, low temperatures and pressure increase lead to a shifting of the equilibrium towards the desired product, methanol. The simultaneously occurring, likewise exothermic, reaction 3 is the so-called “water-gas shift” reaction, in which carbon monoxide is converted into carbon dioxide. In place of a mixture of carbon monoxide, carbon dioxide and hydrogen, which is usually referred to as “synthesis gas”, a gas mixture of carbon dioxide and hydrogen can also be employed as educt. Because of the reversibility of the “water-gas shift” reaction, carbon monoxide can be formed. In particular in the case of a recirculation of unconverted gas constituents (so-called synthesis loop), the accumulation of carbon monoxide can thereby occur although the original educt contained no carbon monoxide at all.

Catalytic reactions on an industrial scale generally run under conditions in which the catalyst employed is increasingly deactivated over the period of use. Deactivation can be effected, for example, due to hydrothermal ageing and/or through poisoning of the catalysts by catalyst poisons in the feed gas stream. Even if the feed gas stream is subjected to a cleaning process before being introduced into the reactor, it typically still contains small quantities of contaminants. Traces of sulfur, chlorine, halogen, iron and nickel compounds and unsaturated hydrocarbons, which may be present in the feed gas stream, act as catalyst poisons and may have a negative impact on the lifetime and thus the methanol production that can be achieved over the lifetime of the catalysts, i.e. the “lifetime yield”, are of particular importance.

For example, it is known that metals can form sulfides even if sulfur is present only in small quantities as a contaminant in the feed stream. This relates in principle to all catalytic methods in which metal or noble metal catalysts are used, such as for example ammoxidation reactions, dehydrogenation reactions, catalytic reforming reactions and oxidation reactions, in particular including methanol synthesis.

Carbonyls such as Fe(CO)₅ or Ni(CO)₄ may be present in particular in the case of methanol synthesis starting from a synthesis gas stream. They can already form as byproducts during the extraction of the synthesis gas from coal or through the reaction of carbon monoxide, which is supplied via the synthesis gas stream or forms within a synthesis loop, with the stainless-steel linings of the pipe walls. During contact with the catalyst for the methanol synthesis-typically a catalyst based on Cu/Zn/Al—these carbonyls are adsorbed thereby and then decompose into CO and the corresponding metal, which drastically reduces the catalytic activity of the catalyst and/or results in an increased formation of undesired byproducts. This can result in the catalyst needing to be replaced sooner. The deactivation of methanol catalysts by iron has been known about for a long time and is described, for example, in Harald H. Kung, Catal. Today, 1992, 11, pp. 443-453. Because of the high gas throughput in the case of catalytic methanol synthesis from synthesis gas, low concentrations, for example in the ppb range, of carbonyls such as Fe(CO)₅ can already be sufficient to significantly reduce the life of the catalyst.

To prevent or reduce such a deactivation of the employed catalysts by catalyst poisons, there can be a cleaning of the synthesis gas upstream of the actual synthesis reaction. Here, cleaning materials (also known as guard bed materials) which are capable of removing possible catalyst poisons by adsorption, absorption and/or decomposition are used.

Typical catalyst poisons in the case of methanol synthesis include, among others, sulfur and chlorine compounds. It is proposed in GB 1 357 335 to provide a guard bed, which contains oxides of alkali metals, alkaline earth metals, manganese, yttrium or lanthanum supported on aluminium oxide, upstream of a “water-gas shift” catalyst based on copper. WO 01/17674 describes a guard bed which contains a chloride absorbent based on lead and a support therefor.

U.S. Pat. No. 5,451,384 A describes the removal of metal carbonyls from a gas stream, for example a synthesis gas stream, through adsorption using lead oxide on a support material. EP 2 069 231 A1 discloses a process for the removal of metal carbonyls from a synthesis gas stream using an adsorbent which contains activated carbon or a hydrophobic zeolite.

WO 2008/144402 describes a process for epoxidizing olefins, in particular ethylene. A reactor system which comprises one or more cleaning zones and a reaction zone is described therein, wherein the reaction zone is arranged inside one or more open reactor tubes of a shell-and-tube heat exchanger reactor vessel. The cleaning zones can be arranged either in a separate reactor vessel upstream of the shell-and-tube heat exchanger reactor vessel or inside the reactor vessel, wherein, in the latter case, the cleaning zone is positioned upstream of the reactor tubes, for example in the headspace of the reactor vessel. In both cases, it is necessary to additionally control the temperature of the cleaning zones independently of controlling the temperature of the reactor tubes, in order to be able to guarantee an optimum temperature for the cleaning action of the absorbent. In addition, not all current reactors have enough room in the headspace of the reactor vessel to place cleaning material therein in sufficient quantity. Moreover, the gas flowing in through the inlet opening of the reactor vessel can lead to turbulence in the absorbent.

In addition to a possible deactivation of the catalyst due to catalyst poisons in the feed gases, there is a further problem in that, when highly active catalyst material is used and there is a correspondingly high conversion of the synthesis gas, a lot of heat is released. The methanol synthesis is therefore generally effected in a shell-and-tube reactor with a heat exchanger, which comprises a large number of reaction tubes, which in each case contain a packed bed of catalyst and are surrounded by a heat exchange fluid, such as a heating or cooling fluid. Nevertheless, when high-activity catalysts are used, it has been shown that a marked non-isothermic reaction zone with marked temperature gradients and a local temperature maximum (hotspot) forms in the initial area of the reactor bed when the reactor is started. This temperature rise and thus the hotspot temperature can be up to 70K above the gas entry temperature or up to 40K above the coolant temperature. In addition, the increased temperature results in a quicker deactivation of the catalyst material due to sintering. The deactivation of the catalyst in turn causes the hotspot to migrate through the reactor. Besides, a high temperature also results in the thermodynamic equilibrium of the reaction being reached. When the thermodynamic equilibrium is reached, no further conversion of the synthesis gas takes place. In the downstream part of the catalyst bed there are then lower temperatures and a falling proportion of carbon oxides in the synthesis gas, since a portion of the oxides has already been converted.

An object of the present invention is therefore to provide a method for producing methanol and to develop reactors used herein such that the above-described disadvantages are prevented or at least reduced, and the space-time yield can be increased or at least maintained. In particular, the object of the present invention is to provide a method for producing methanol and reactors used therefor, whereby it is made possible to increase the lifetime of a methanol catalyst in the presence of catalyst poisons and hydrothermal ageing at the same time as maintaining, and ideally even increasing, the methanol yield.

The inventors of the present invention have surprisingly established that this object is achieved by providing an upstream cleaning layer and at least one downstream catalyst layer inside the reactor tubes in a tube reactor with a heat exchanger. Through this arrangement, the temperature of the supplied synthesis gas can be pre-heated via the heat exchange liquid. The pre-heating of the supplied synthesis gas has a positive effect both on the cleaning action of the cleaning layer and on the conversion of the synthesis gas in a downstream catalyst layer. The advantages of the present invention also arise when a reactor with one or more thermoplates is used in place of a tube reactor with a heat exchanger. Such reactors are described in EP 1 147 807 A, EP 3 401 299 A1 and DE 10 2004 036 695 A1, for example. The reaction is carried out in the interspaces between the thermoplates of such a reactor, which are filled with solid catalyst in the state of the art. The above-described advantages of the present invention can also be realized through the provision according to the invention of an upstream cleaning layer and at least one downstream catalyst layer inside the interspaces between thermoplates.

### SUMMARY OF THE INVENTION

The invention relates to a reactor for the catalytic production of methanol comprising two or more than two reaction zones, which are surrounded by a heat exchange fluid, wherein at least one catalyst layer is arranged in each of the two or the more than two reaction zones, and at least one cleaning layer, which comprises a cleaning material, is arranged in each of the two or the more than two reaction zones, and wherein the at least one cleaning layer is arranged upstream of the at least one catalyst layer. The reaction zones are preferably reactor tubes.

The invention also relates to a method for the catalytic production of methanol from synthesis gas, wherein the method comprises the following steps:
(a) providing a reactor, preferably a heat exchanger reactor,
(b) arranging two or more than two reaction zones in the reactor,
(c) charging the reactor with synthesis gas, comprising hydrogen and carbon oxides,
(d) converting the synthesis gas in the reactor to methanol under methanol synthesis conditions, and
(e) conducting the generated methanol and the unconverted synthesis gas out of the reactor,
wherein at least one catalyst layer is arranged in each of the two or the more than two reaction zones, and
wherein at least one cleaning layer, which comprises a cleaning material, is arranged in each of the two or the more than two reaction zones, and wherein the at least one cleaning layer is arranged upstream of the at least one catalyst layer.

### DETAILED DESCRIPTION OF THE INVENTION

In a first embodiment, the invention relates to a reactor for the catalytic production of methanol. The reactor comprises two or more than two reaction zones, which are surrounded by a heat exchange fluid. The reaction zones are preferably formed by reactor tubes.

Alternatively, the reaction zones can also be formed using thermoplates, through which a heat exchange fluid flows. Within the meaning of the present invention a thermoplate consists of two metal sheets which are joined, preferably welded together, at the edges and over the surface of which a large number of additional joins, preferably spot welds, which likewise join the metal sheets to each other, is distributed. Such metal sheets can be produced in an automated manner by robots or machines and thus at very favourable prices. After the welding, the thermoplates obtained are expanded by a hydraulic forming, as a rule by injecting a liquid under high pressure, as a result of which cushion-like channels through which a heat exchange fluid, such as a heating or cooling fluid, can be conducted form between the metal sheets forming the thermoplates. Via the heat transporting spaces, heat energy can therefore be both supplied to and removed from particular areas of the reactor by conducting heat exchange fluid through. For this, to begin with, three or more than three thermoplates are arranged substantially parallel to each other and to the longitudinal direction in a reactor. Within the meaning of the invention, substantially parallel means that the alignment of the thermoplates with respect to each other and to the longitudinal direction deviates from each other from the parallel by +/−20° or less, preferably by +/−10° or less, particularly preferably by +/−5° or less, quite particularly preferably by +/−2° or less. In this embodiment, the interspaces or free spaces between in each case two neighbouring thermoplates form the reaction zones.

Within the meaning of the present invention, a “reaction zone” can therefore be designed as a reactor tube or as an interspace between two neighbouring thermoplates. The reaction zones are preferably formed as reactor tubes.

At least one catalyst layer and at least one cleaning layer are arranged in each of the two or more than two reaction zones. The cleaning layer comprises a cleaning material and is arranged upstream in the reaction zones. The catalyst layer(s) is/are arranged downstream in the reaction zones.

In the present invention, the terms “upstream” or “downstream” denote the arrangement of the cleaning and catalyst layers in the reactor. A gas inlet side and a gas outlet side for synthesis gas are provided in the reactor. The synthesis gas enters the interior of the reactor, in which the catalyst layers are provided, through the gas inlet side. After flowing through the catalyst layers, the synthesis gas exits the reactor on the gas outlet side. The stream of synthesis gas thus defines a direction, wherein the synthesis gas enters the interior of the reactor through the “upstream” gas inlet side and exits the interior of the reactor through the “downstream” gas outlet side. In the reactor, the cleaning layer is thus closer to the gas inlet side than the first catalyst layer, which is closer to the gas outlet side of the reactor.

In a preferred embodiment, the reactor comprises 2 to 25,000 reaction zones, more preferably 1000 to 20,000 reaction zones, in particular 2500 to 15,000 reaction zones.

The reactor is preferably formed as a shell-and-tube reactor which contains 2 to 25,000 reactor tubes, more preferably 1000 to 20,000 reactor tubes, in particular 2500 to 15,000 reactor tubes.

In a further embodiment, the reactor is formed using thermoplates, wherein the reactor contains 3 to 10,000 thermoplates, preferably 100 to 8000 thermoplates, in particular 500 to 5000 thermoplates.

The reaction zones are preferably arranged substantially parallel to the central longitudinal axis of the reactor.

In a preferred embodiment, water or water vapour, a heat transfer oil, such as Dowtherm A, or a suitable gas is employed as heat exchange fluid. Water or water vapour is preferably used as heat exchange fluid or coolant fluid. In other words, the reactor is preferably formed as a water-cooled, oil-cooled or as a gas-cooled reactor, in particular as a water-cooled reactor.

The reactor tubes can have an internal diameter in the range of from 10 to 80 mm, preferably in the range of from 20 to 75 mm and particularly preferably in the range of from 25 to 70 mm. The reactor tubes can have a length in the range of from 0.5 to 25 m, preferably in the range of from 2 to 15 m and particularly preferably in the range of from 5 to 10 m.

In the embodiment in which the reaction zones are formed using thermoplates, the distances between in each case two thermoplates are typically in the range of from 10 mm to 100 mm, preferably in the range of from 20 to 50 mm. Here, the distance relates to the distance from centre line to centre line, wherein the centre line denotes the axis of symmetry of the thermoplates formed of two metal sheets. The distance can be adapted to the dimensions of the catalyst particles in order to ensure an optimum removal of heat and a good bulk material behaviour when the catalyst is being filled and emptied.

At least one catalyst layer which comprises one or more catalysts is arranged in each of the reaction zones, which are formed as reactor tubes in a preferred embodiment. Two or more catalyst layers can also be arranged in the reaction zones. The catalyst layers are preferably arranged directly adjacent to each other in the flow direction of the synthesis gas.

In a preferred embodiment, at least two catalyst layers that are different from each other are arranged in each of the two or more than two reaction zones, which are preferably reactor tubes. The first catalyst layer is arranged upstream and the second catalyst layer is arranged downstream. The activity of the first catalyst layer preferably differs from the activity of the second catalyst layer.

Here, the activity of a catalyst layer denotes the degree of conversion of educts to products in the catalyst layer, standardized to the height of the catalyst layer. The activity of a first catalyst layer is therefore lower than the activity of a second catalyst layer that differs from it if the degree of conversion of educts to products of the first catalyst layer is lower than that of the second catalyst layer when the two catalyst layers are the same height (i.e. have the same bulk volume) and the reaction conditions are the same.

The activity of a catalyst can be influenced, for example, by the chemical composition of the catalyst material, such as by adding an activity-moderating promoter, by varying the available surface area etc. The catalysts are preferably solid. The catalyst layers can be formed of catalysts in powdered form and/or of catalysts in the form of moulded bodies, in particular pressed moulded bodies, such as for example pressed tablets or pressed rings, pellets, or of extrudates.

The moulded bodies can have different geometries, such as spheres, cylinders or hollow bodies such as rings. Spherical pellets can have a diameter d of from 1 mm to 12 mm, preferably from 2 mm to 10 mm and particularly preferably from 3 mm to 8 mm. Extrudates, cylindrical pellets or pressed tablets or rings can have a diameter d of from 1 mm to 12 mm, preferably from 2 mm to 10 mm and particularly preferably from 3 mm to 8 mm and a height h of from 1 mm to 10 mm, preferably from 2 mm to 8 mm and particularly preferably from 3 mm to 6 mm.

The geometry and size of pellet catalysts can be the same or different in different catalyst layers.

For example, a larger quantity of educt can be converted to product in a catalyst layer which is formed of pellets in the form of small spheres than in a corresponding catalyst layer of the same material with the same height which is formed of pellets in the form of larger spheres.

In order to implement different activities, smaller cylindrical pellets, for example with a 3 mm diameter and a 3 mm height, can be used in one catalyst layer, while larger cylindrical pellets of the same material, for example with a 6 mm diameter and a 4 mm height, can be employed in another catalyst layer. In one embodiment, smaller cylindrical pellets with a 3 mm diameter and a 3 mm height are used in one catalyst layer, while larger cylindrical pellets of the same material with a 6 mm diameter and a 4 mm height are employed in another catalyst layer.

The catalyst layers are preferably formed of catalysts in pellet form, wherein it is particularly preferred that the pellet size of the catalysts in different catalyst layers differs in particular for catalyst layers with different activities.

In one embodiment, the catalysts of the individual catalyst layers have different compositions. For example, the catalysts of the individual catalyst layers can differ in terms of the presence or absence of promoters or the quantities thereof.

The catalysts are preferably arranged in the form of fixed packed beds. The catalyst layers are formed by loading the desired catalyst layers into the reactor tubes one after another. The catalyst layers are thereby directly adjacent to each other. During the loading it is ensured that the catalyst layers are arranged one after another in the flow direction of the synthesis gas.

In a preferred embodiment, the activity of the first catalyst layer is lower than the activity of the further catalyst layer or than the activity of the further catalyst layers. Alternatively to this it can also be provided that the activity of the first catalyst layer is higher than the activity of the second catalyst layer, and the activity of the further catalyst layer(s), if present, increases successively in each case.

In a particularly preferred embodiment, one or more further catalyst layers, preferably two or more than two further catalyst layers, are arranged in the reactor tubes, wherein the further catalyst layers are arranged in each case downstream of the second catalyst layer and wherein the activity of the further catalyst layers increases successively towards the downstream end of the reactor.

The layer height of the first catalyst layer is preferably 20 to 80%, relative to the total height of all cleaning layers and catalyst layers in the reaction zone. As service life increases, a shrinkage of catalysts and thus a reduction in the height of the catalyst layer(s) can occur. The specified relative layer heights of the catalyst layers are to be understood to relate to the initial state of the reaction zones loaded with cleaning material and catalyst, i.e. to the state before the reactor is started up.

Copper-based methanol synthesis catalysts, in particular Cu/Zn/Al catalysts, can be used as catalysts.

The copper-based methanol synthesis catalysts used can have different copper dispersions and different levels of activity. Here, the dispersion is a measure of the particle size of the copper particles present in the catalyst and can be determined by CO chemisorption and subsequent methanation of the metal-bonded CO, for example.

At least one cleaning layer, which comprises one or more cleaning materials, is arranged in each of the reaction zones, which are preferably reactor tubes. The at least one cleaning layer is arranged upstream of the at least one catalyst layer. Two or more cleaning layers can also be arranged in the reaction zones, which are preferably reactor tubes. The cleaning layer(s) are preferably arranged directly adjacent to each other and adjacent to the at least one catalyst layer in the flow direction of the synthesis gas.

In a preferred embodiment, an additional cleaning layer is arranged upstream, i.e. outside the two or more than two reaction zones, which are preferably reactor tubes. The optional additional cleaning layer is preferably arranged inside the reactor. The optional additional cleaning layer contains a cleaning material which is the same as or different from the cleaning material in the two or the more than two reaction zones, which are preferably reactor tubes.

The layer height of the cleaning layer(s) in the reaction zones, which are preferably reactor tubes, is preferably 5 to 30%, preferably 5 to 20%, more preferably 7.5 to 20%, particularly preferably 10 to 15%, relative to the total layer height of all cleaning layer(s) and catalyst layers in the reaction zone. The specified relative layer heights of the cleaning layer(s) are to be understood to relate to the initial state of the reaction zones loaded with cleaning material and catalyst, in particular the initial state of the reactor tubes loaded with cleaning material and catalyst, i.e. to the state before the reactor is started up.

The cleaning material is characterized in that it is capable of reducing the quantity of contaminants which are present in a synthesis gas stream, and which can poison the catalyst used under the prevailing conditions.

At the start of being used in the reactor, the cleaning layer(s) are preferably capable of reducing the quantity of reductions from the synthesis gas stream, relative to the quantity thereof in the synthesis gas stream employed, by 10 percent by weight or more, more preferably by 25 percent by weight or more, particularly preferably by 50 percent by weight or more, quite particularly preferably by 90 percent by weight or more. At the start of being used in the reactor, the cleaning layer(s) are preferably capable of reducing the quantity of reductions from the synthesis gas stream, relative to the quantity thereof in the synthesis gas stream employed, by 1 percent by weight or more, more preferably by 5 percent by weight or more, particularly preferably by 25 percent by weight or more, quite particularly preferably by 40 percent by weight or more.

The contaminants, the quantity of which in the synthesis gas stream is reduced by the cleaning material, are in particular metals and compounds thereof, such as elemental iron, iron-containing compounds, in particular iron carbonyls, and nickel-containing compounds, in particular nickel carbonyls, sulfur-containing compounds, such as sulfides or sulfates, unsaturated hydrocarbons, chlorine-containing compounds, and mixtures thereof.

The reduction of the quantity of contaminants can be effected via an adsorption and/or absorption of the contaminants on the cleaning material and/or by a decomposition of the contaminants. By adsorption within the meaning of the present invention is meant that substances, in particular molecules, stick on the surface of the cleaning agent and accumulate on the accessible surface thereof, wherein the adhesion is effected substantially not via chemical bonds but via van der Waals forces. In contrast, the term absorption denotes the process in which an adhesion of substances, in particular of molecules, to the accessible surface of the cleaning agent is effected through the formation of chemical bonds.

A reduction of the quantity of contaminants through decomposition by the cleaning material includes the case that, during the decomposition of the contaminants, products form which, under the prevailing conditions, for their part only function to a lower degree, preferably to a low degree, in particular no longer function at all as a catalyst poison for the catalysts used. A reduction of the quantity of contaminants through the decomposition thereof by the cleaning material also includes the case that, during the decomposition of the contaminants, products form which can be bonded to the cleaning material via adsorption and/or absorption.

The cleaning material preferably has an intake capacity for contaminants of up to 1 percent by weight, more preferably up to 5 percent by weight, particularly preferably up to 25 percent by weight, quite particularly preferably up to 40 percent by weight, of the contaminants, relative to the weight of cleaning material used. The cleaning material preferably has an intake capacity for sulfur-containing contaminants, relative to the weight of cleaning material used, of 500 ppmw or more. The cleaning material preferably has an intake capacity for heavy-metal-containing contaminants, relative to the weight of cleaning material used, of 500 ppmw or more. The intake capacity can be determined in a breakthrough test, e.g. via the mass balance of the inlet stream and the outlet stream. Here, for example an inlet stream with a known quantity of sulfur contaminant is conducted over the cleaning material until a significant quantity of sulfur contaminants is detected in the outlet stream. This is also called breakthrough. The quantity of sulfur over a given time period can be calculated from the volume which is conducted over the cleaning material for each unit of time. This method can be applied in a corresponding manner to other contaminants, such as heavy metal contaminants or comparable contaminants.

Suitable cleaning materials can preferably be selected from the group consisting of zinc-based cleaning materials, such as ZnO, aluminium-based cleaning materials, such as aluminium oxide, preferably Al₂O₃, silicon-based cleaning materials, such as silicon dioxide, AlSiOx-based cleaning materials, such as zeolites, activated carbon, clays, and copper-based cleaning materials. Oxides of alkali metals, alkaline earth metals, manganese, yttrium or lanthanum supported on aluminium oxide are also suitable for example for reducing the quantity of catalyst poisons, in particular of sulfur and chlorine compounds. Cleaning materials based on activated carbon or zeolites are described, for example, in EP 2 069 231 B1 for the removal of metal carbonyls from a synthesis gas stream.

In principle, the above-described methanol synthesis catalysts can also be used as cleaning materials after a complete or partial deactivation. This includes, for example, methanol synthesis catalysts which have already been used in a methanol synthesis reactor and have hereby lost at least some of their activity. The cleaning materials preferably comprise at most 50 wt.-%, preferably at most 10.0 wt.-%, more preferably at most 2.0 wt.-%, in particular 0.1 wt.-% or less, of methanol synthesis catalysts after a complete or partial deactivation, relative to the total weight of the cleaning materials present in the cleaning layers.

In a preferred embodiment, no methanol synthesis catalysts are used as cleaning materials after a partial deactivation. In this embodiment, the cleaning material is substantially inert in relation to the methanol synthesis, in other words it does not contribute significantly to the catalytic production of methanol. Preferably, 5 percent by volume or less, more preferably 1 percent by volume or less, in particular 0.1 percent by volume or less, of methanol is formed by the cleaning material, relative to the total quantity of methanol formed per unit of time, measured at the gas outlet.

Preferably, 5 percent by volume or less of byproducts, preferably 1 percent by volume or less of byproducts, particularly preferably 0.1 percent by volume or less of byproducts is formed by the cleaning material, relative to the total quantity of gas at the gas outlet. Possible byproducts include dimethyl ether, higher alcohols or higher hydrocarbons.

The cleaning layers can be formed of cleaning material in powdered form and/or of cleaning material in the form of moulded bodies, in particular pressed moulded bodies, such as for example pressed tablets or pressed rings, pellets, or of extrudates. The cleaning layers are preferably formed of cleaning material in pellet form. The cleaning material in the cleaning layer(s) is preferably arranged in the form of fixed packed beds.

A method for the catalytic production of methanol from synthesis gas is also a subject-matter of the invention. The method has the following steps:
(a) providing a reactor, preferably a heat exchanger reactor;
(b) arranging two or more than two reaction zones in the reactor,
(c) charging the reactor with synthesis gas, comprising hydrogen and carbon oxides,
(d) converting the synthesis gas in the reactor to methanol under methanol synthesis conditions, and
(e) conducting the generated methanol and the unconverted synthesis gas out of the reactor,
wherein at least one catalyst layer is arranged in each of the two or the more than two reaction zones, and
wherein at least one cleaning layer, which comprises a cleaning material, is arranged in each of the two or the more than two reaction zones, and wherein the at least one cleaning layer is arranged upstream of the at least one catalyst layer.

The synthesis gas usually comprises a mixture of carbon monoxide, carbon dioxide and hydrogen.

Alternatively to this, a mixture of carbon dioxide and hydrogen can also be used as synthesis gas in the present method for the catalytic production of methanol. In this case, the carbon oxides in the synthesis gas are formed substantially, i.e. 99 vol.-% or more, preferably 99.9 vol.-% or more, relative to the total volume of all carbon oxides in the synthesis gas stream, in particular exclusively, of carbon dioxide. Because of the reversibility of the “water-gas shift” reaction, carbon monoxide can be formed in the reactor, with the result that a mixture of carbon monoxide, carbon dioxide and hydrogen is formed in situ in the reactor. In particular in the case of a recirculation of unconverted gas constituents (so-called synthesis loop), the accumulation of carbon monoxide, which can be fed back into the reactor in this way, can thereby occur.

In a preferred embodiment, the reaction zones are designed as reactor tubes.

At least two catalyst layers that are different from each other are preferably arranged in each of the two or the more than two reaction zones, which are preferably reactor tubes, wherein the first catalyst layer is arranged upstream and the further catalyst layer(s) is/are arranged downstream. In a particularly preferred embodiment, the activity of the first catalyst layer is lower than the activity of the further catalyst layer(s).

Water or water vapour is preferably employed as heat exchange fluid. The reactor preferably used in the method is a water-cooled reactor. The cooling temperature is preferably in the range of from 180° C. to 270° C., preferably in the range of from 200° C. to 270° C., more preferably in the range of from 210° C. to 260° C., in particular in the range of from 220° C. to 250° C.

While the synthesis gas is flowing through the cleaning layer in the reaction zones, which are preferably reactor tubes, it is pre-heated before entering the (first) catalyst layer. The degree of pre-heating is dependent on the entry temperature of the synthesis gas, the temperature of the heat exchange fluid and the layer thickness of the cleaning layer(s).

The entry temperature of the synthesis gas at the gas inlet of the reactor is usually in the range of from 150° C. to 300° C., preferably in the range of from 170° C. to 270° C.

In a preferred embodiment, the temperature of the synthesis gas is increased by a value in the range of from 0.1K to 50K, preferably in the range of from 5K to 40K, in particular in the range of from 10K to 30K, through the use of a cleaning layer in the reaction zones.

In a preferred embodiment, the temperature of the synthesis gas pre-heated through the use of a cleaning layer in the reaction zones is in the range of from 180° C. to 260° C., preferably in the range of from 200° C. to 260° C., more preferably in the range of from 210° C. to 250° C., in particular in the range of from 220° C. to 240° C.

The invention is described in more detail below using several examples with reference to the appended figures. The figures show:

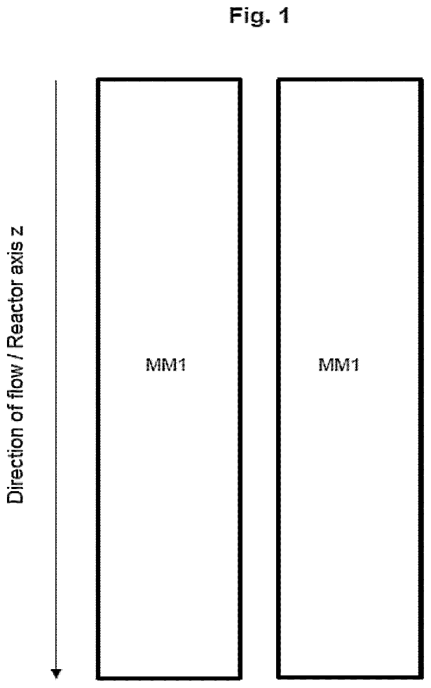

FIG. 1 illustrative first arrangement, not according to the invention, of two reactor tubes without a cleaning layer, with a catalyst layer MM1

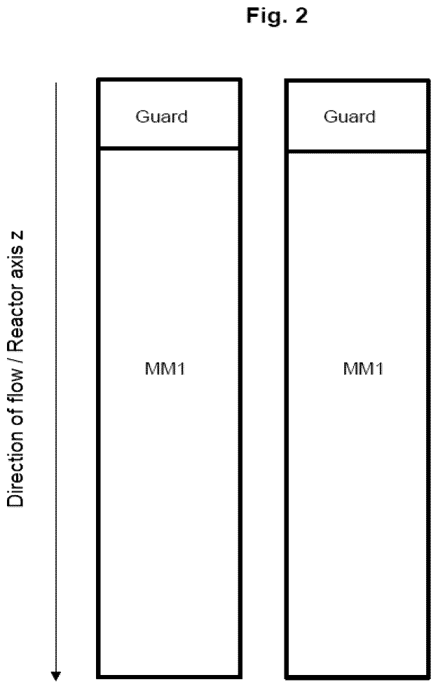

FIG. 2 illustrative second arrangement, according to the invention, of two reactor tubes with a cleaning layer and a catalyst layer MM1

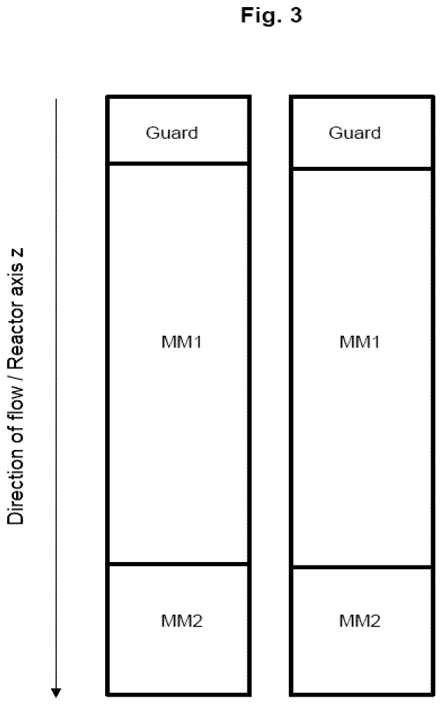

FIG. 3 illustrative third arrangement, according to the invention, of two reactor tubes with a cleaning layer and two catalyst layers MM1 and MM2

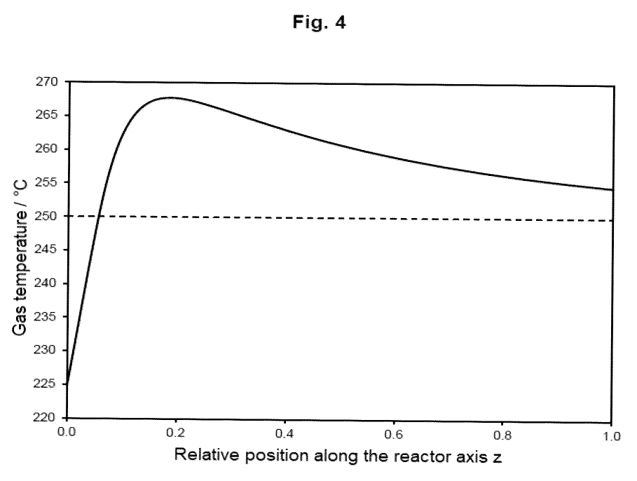

FIG. 4 example temperature profile for an arrangement, not according to the invention, without a cleaning layer, with a catalyst layer MM1

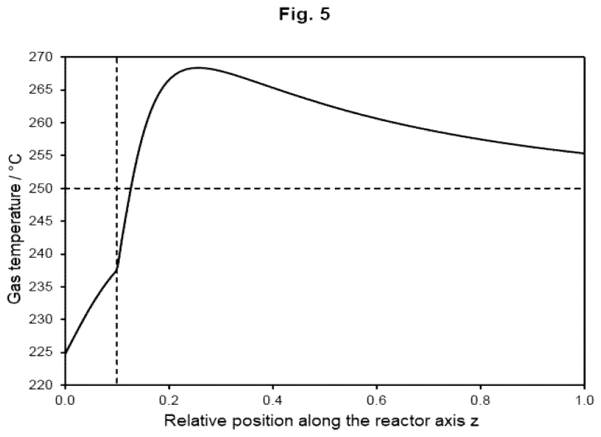

FIG. 5 example temperature profile for an arrangement, according to the invention, with a cleaning layer and a catalyst layer MM1, wherein the vertical dashed line symbolizes the boundary between cleaning layer and catalyst layer MM1, and the horizontal dashed line shows the temperature of the heat exchange fluid

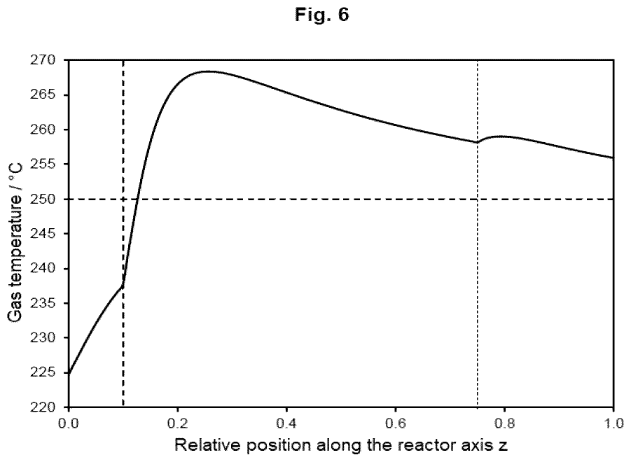

FIG. 6 example temperature profile for an arrangement, according to the invention, with a cleaning layer and two catalyst layers MM1 and MM2, wherein the two vertical dashed lines symbolize the boundaries between cleaning layer and catalyst layer MM1 and between catalyst layer MM1 and catalyst layer MM2, and the horizontal dashed line shows the temperature of the heat exchange fluid

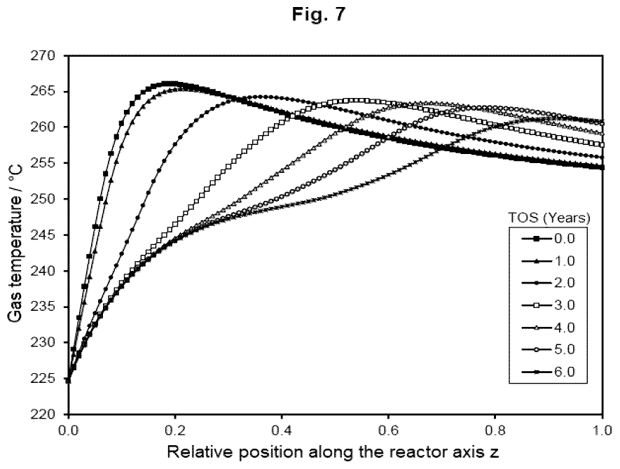

FIG. 7 example temperature profiles for an arrangement, not according to the invention, without a cleaning layer, with a catalyst layer MM1 for various service lives

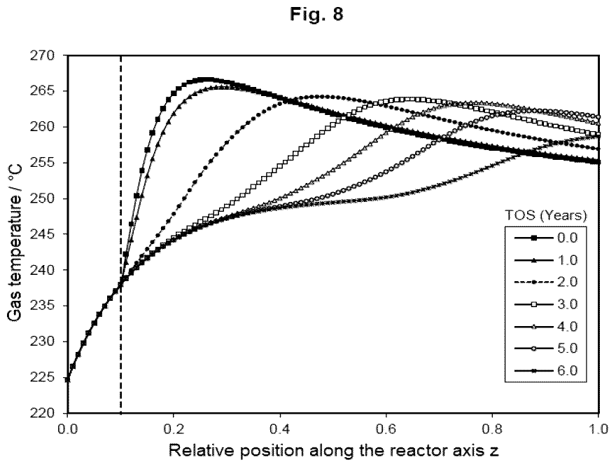

FIG. 8 example temperature profiles for an arrangement, according to the invention, with a cleaning layer and a catalyst layer MM1 for various service lives

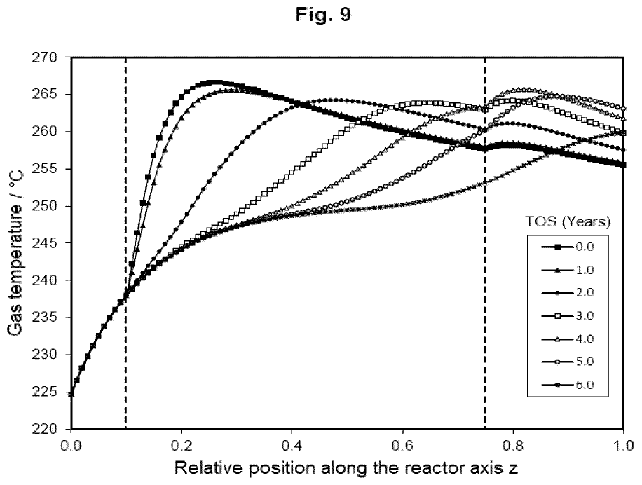

FIG. 9 example temperature profiles for an arrangement, according to the invention, with a cleaning layer and two catalyst layers MM1 and MM2 for various service lives

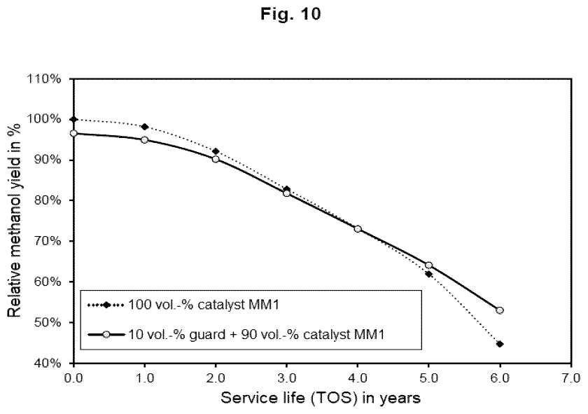

FIG. 10 example development of the methanol yield as a function of the service life for an arrangement, according to the invention, with a cleaning layer and a catalyst layer MM1 compared with an arrangement, not according to the invention, with a catalyst layer MM1 but without a cleaning layer, assuming a deactivation slowed by 20% due to the cleaning layer (standardized to the initial methanol yield of the arrangement, not according to the invention, with a catalyst layer MM1 but without a cleaning layer)

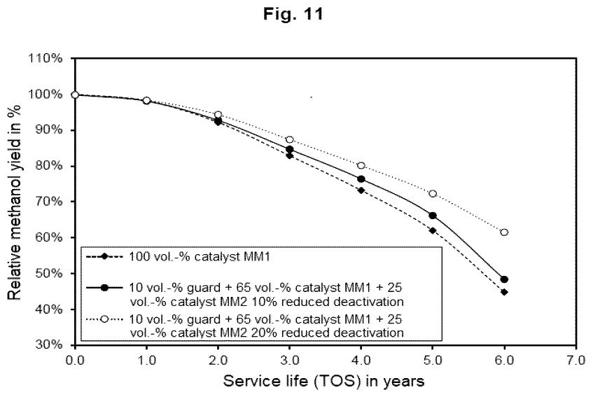

FIG. 11 example development of the methanol yield as a function of the service life for an arrangement, according to the invention, with a cleaning layer and two catalyst layers MM1 and MM2 compared with an arrangement, not according to the invention, with a catalyst layer MM1 but without a cleaning layer, assuming a deactivation slowed by 10% and by 20% due to the cleaning layer (standardized to the initial methanol yield of the arrangement, not according to the invention, with a catalyst layer MM1 but without a cleaning layer)

### EXAMPLES

In the following examples, a copper-based catalyst from the MegaMax® series is used in different pellet sizes and referred to as MM1 and MM2, respectively, in the examples and the figures. MM1 has a pellet size of 6 mm×4 mm in a cylindrical shape (diameter×height) and MM2 has a pellet size of 3 mm×3 mm in a cylindrical shape (diameter×height).

The test arrangement on which the simulations are based was chosen so that it reflects the setup of an arrangement of a methanol synthesis plant typical in industry. The reactor is formed as a water-cooled reactor (WCR) and synthesis gas flows through it at a space velocity of approx. 14,000 h⁻¹.

In the following examples and figures, TOS (time-on-stream) furthermore refers to the service life of a catalyst in years, and z is the relative position along the reactor axis in the reactor tubes in the flow direction, assuming a 100% fill, wherein the gas inlet is at z equals 0.0 and the gas outlet is at z equals 1.0. In the figures, the dashed line represents the cooling temperature. The vertical dotted lines illustrate the different plies.

### Example 1 (Comparison)

A water-cooled methanol reactor with a loading of 100 vol.-% of a packed bed of catalyst MM1 without an upstream cleaning layer is used in Example 1. This corresponds to the setup depicted in FIG. 1.

### Example 2

A water-cooled methanol reactor with a loading with two layers (10 vol.-% guard bed, 90 vol.-% packed bed of catalyst MM1) is used in Example 2. This corresponds to the setup depicted in FIG. 2.

### Example 3

A water-cooled methanol reactor with a loading with three layers (10 vol.-% guard bed, 65 vol.-% packed bed of catalyst MM1 and 25 vol.-% packed bed of catalyst MM2) is used in Example 3. This corresponds to the setup depicted in FIG. 3.

### Example 4

FIG. 4 shows a typical temperature profile in the water-cooled methanol reactor according to Example 1 with a cooling temperature of 250° C. as a function of the position along the reactor axis z.

FIG. 5 shows a typical temperature profile in the water-cooled methanol reactor according to Example 2 with a cooling temperature of 250° C. as a function of the position along the reactor axis z. The guard bed was assumed to be a packed bed of spheres with a diameter of 3 mm that is inert with regard to the methanol production.

FIG. 6 shows a typical temperature profile in the water-cooled methanol reactor according to Example 3 as a function of the position along the reactor axis z.

The comparison of FIG. 4 with FIG. 5 shows that the presence of a cleaning layer arranged upstream leads to a significant heating of the entering gas from approx. 225° C. to approx. 238° C., i.e. by approximately 13K. Despite this heating, the hotspot temperature only increases by less than 1K, from approx. 267° C. to approx. 268° C. Through the heating-up of the gas in the guard bed layer (inert for the methanol reaction), the synthesis gas reaches the catalyst at a higher temperature that is advantageous for the activity thereof.

In FIG. 6, a second hotspot can additionally be seen in the second (more active) catalyst layer. The hotspot is the result of the exothermicity forming due to the additional reaction and occurring due to the higher activity of the second catalyst layer, and thus illustrates a further increase in the yield compared with Example 2 in FIG. 5. The comparison of FIG. 6 with FIG. 5 shows that the presence of a second more active catalyst layer leads to a heating of the gas by approximately 2K, which results from a higher rate of conversion by this catalyst.

### Example 5

FIG. 7 shows typical temperature profiles in the water-cooled methanol reactor according to Example 1 as a function of the position along the reactor axis z for service lives (TOS=time-on-stream) of from 0 to 6 years. The underlying deactivation profile was derived from real plant data. The deactivation proceeds along the reactor axis z over time and is described mathematically using a so-called logistic function. This leads to the displacement of the hotspot, observed in real plants, downstream over the service life.

FIG. 8 shows typical temperature profiles in the water-cooled methanol reactor according to Example 2 as a function of the position along the reactor axis z for service lives (TOS=time-on-stream) of from 0 to 6 years. The guard bed was assumed in each case to be a packed bed of spheres with a diameter of 3 mm that is inert with regard to the methanol production. The same deactivation behaviour as in FIG. 7 was used as the basis in the simulation (i.e. a reduced ageing to be expected because of the reduction in the quantity of catalyst poisons in the synthesis gas due to the guard bed was not taken into consideration). The simulations therefore reflect a “worst case” scenario and merely demonstrate the positive influence of the combination according to the invention of guard bed layer and catalyst layering on the temperature profile in the reactor tube over the lifetime.

The comparison of FIG. 7 and FIG. 8 shows, as already previously, that for one thing the presence of a cleaning layer arranged upstream contributes to a significant heating of the entering gas from approx. 225° C. to approx. 238° C., i.e. by approximately 13K. Despite this heating, the hotspot temperature only increases by less than 1K, from approx. 267° C. to approx. 268° C. Through the heating-up of the gas in the guard bed layer (inert for the methanol reaction), the synthesis gas reaches the catalyst at a higher temperature that is advantageous for the activity thereof.

In FIG. 9, a second hotspot can again be seen in the second (more active) catalyst layer. The comparison of FIG. 9 with FIG. 8 shows that the presence of the second catalyst layer with a more active catalyst still leads, for example in the case of a service life of 2 years, to a significant heating of the gas by approximately 3K, because of a higher rate of conversion by the more active catalyst.

### Example 6

FIG. 10 shows the relative methanol yield in percent as a function of the service life for an arrangement according to the invention with two layers according to Example 2 (10 vol.-% guard bed and 90 vol.-% of a packed bed of catalyst MM1) compared with an arrangement not according to the invention without an upstream cleaning layer according to Example 1 (100 vol.-% packed bed of catalyst MM1). The values are standardized to the methanol yield of a 100 vol.-% packed bed of catalyst MM1 at the time point 0 years. A deactivation reduced by 20% because of the reduction in the quantity of catalyst poisons in the synthesis gas due to the cleaning material in the guard bed was assumed in the simulations for the arrangement according to the invention. FIG. 10 shows a slower decline in the methanol yield for the arrangement according to the invention. This difference between the reactor according to the invention and a conventional reactor arises in particular from an operating time of 4 years, from which a much higher methanol yield manifests itself.

### Example 7

FIG. 11 shows the relative methanol yield in percent as a function of the service life for an arrangement according to the invention with three layers according to Example 3 (10 vol.-% guard bed, 65 vol.-% packed bed of catalyst MM1 and 25 vol.-% packed bed of catalyst MM2) compared with an arrangement not according to the invention without an upstream cleaning layer according to Example 1 (100 vol.-% packed bed of catalyst MM1). The values are standardized to the methanol yield of a 100 vol.-% packed bed of catalyst MM1 at the time point 0 years. A deactivation reduced by 10% and another reduced by 20% because of the reduction in the quantity of catalyst poisons in the synthesis gas due to the cleaning material in the guard bed was assumed in the simulations for the arrangement according to the invention. FIG. 11 shows that an initial yield of methanol identical to the comparison example is achieved through the arrangement of catalyst layers with different activity levels, although only 90% of the reactor is filled with catalyst (this therefore corresponds to a higher space-time yield), with a cleaning layer being introduced at the same time. From the beginning and over the entire lifetime, the slower decline in the methanol yield for the arrangement according to the invention leads to a higher methanol yield compared with an arrangement not according to the invention.

The above-described example embodiments are not to be understood as limitative. Other embodiments which are consistent with the above-described example embodiments are now described in an obvious manner for a person skilled in the art.

1. A reactor for the catalytic production of methanol, comprising two or more reaction zones, which are surrounded by a heat exchange fluid,
wherein at least one catalyst layer is arranged in each of the two or more reaction zones,
wherein at least one cleaning layer, which comprises a cleaning material, is arranged in each of the two or more reaction zones, and
wherein the at least one cleaning layer is arranged upstream of the at least one catalyst layer.

2. The reactor of claim 1, wherein
at least two catalyst layers that are different from each other are arranged in each of the two or more reaction zones, and
wherein, of the at least two catalyst layers, a first catalyst layer is arranged upstream and a further catalyst layer(s) is/are arranged downstream.

3. The reactor of claim 2, wherein an activity of the first catalyst layer is lower than an activity of the further catalyst layer(s).

4. The reactor of claim 1, wherein a layer height of the cleaning layer(s) is 5 to 30% of a total layer height of all catalyst layer(s) and cleaning layer(s) in the reaction zones.

5. The reactor of claim 1, wherein an additional cleaning layer is arranged upstream of the two or more than two reaction zones, and
wherein the additional cleaning layer contains cleaning material that is the same as or different from the cleaning material in the two or more reaction zones.

6. The reactor of claim 1, wherein the cleaning material essentially does not contribute to the catalytic production of methanol.

7. The reactor of claim 1, wherein the cleaning material reduces a quantity of contaminants present in a synthesis gas stream introduced into the reactor,
wherein the contaminants can poison catalysts in the catalyst layer(s) and are reduced by adsorption, absorption, and/or decomposition.

8. The reactor of claim 1, wherein catalysts in the catalyst layer(s) and/or the cleaning material in the cleaning layer(s) are arranged in the form of fixed packed beds.

9. The reactor of claim 1, wherein the two or more reaction zones are arranged in a heat exchanger reactor,
wherein the reaction zones are arranged substantially parallel and/or radial with respect to a central longitudinal axis of the reactor, and
wherein the reactor is a water-cooled reactor, an oil-cooled reactor, or a gas-cooled reactor.

10. The reactor of claim 1, wherein the reaction zones are formed as reactor tubes.

11. A method for the catalytic production of methanol from synthesis gas, the method comprising:
providing a reactor;
arranging two or more reaction zones, selected from reactor tubes or reactor plates, which are surrounded by a heat exchange fluid, in the reactor,
charging the reactor with a synthesis gas comprising hydrogen and carbon oxides,
converting the synthesis gas in the reactor to methanol under methanol synthesis conditions, and
conducting the generated methanol and the unconverted synthesis gas out of the reactor,
wherein at least one catalyst layer is arranged in each of the two or more reaction zones,
wherein at least one cleaning layer, which comprises a cleaning material, is arranged in each of the two or more reaction zones, and
wherein the at least one cleaning layer is arranged upstream of the at least one catalyst layer.

12. The method of claim 11, wherein at least two catalyst layers that are different from each other are arranged in each of the two or more reaction zones, and
wherein, of the at least two catalyst layers, a first catalyst layer is arranged upstream and a further catalyst layer(s) is/are arranged downstream.

13. The method of claim 12, wherein an activity of the first catalyst layer is lower than an activity of the further catalyst layer(s).

14. The method of claim 11, wherein the reactor is a water-cooled reactor, and a cooling temperature is in a range of from 180° C. to 270° C.
